(19) United States
(12) Patent Application Publication
(10) Pub. No.: US 2012/0065687 A1
(43) Pub. Date: Mar. 15, 2012

(54) Multi-Radius Vertebral Rod With a Varying Stiffness

(75) Inventors: Rodney R. Ballard, Lakeland, TN (US); Julien J. Prevost, Memphis, TN (US); Christopher F. Scifert, Bartlett, TN (US)

(73) Assignee: Warsaw Orthopedic, Inc., Warsaw, IN (US)

(21) Appl. No.: 12/879,620

(22) Filed: Sep. 10, 2010

(51) Int. Cl. A61B 17/70; A61B 17/88

(52) U.S. Cl. 606/259; 606/279

(57) ABSTRACT

Vertebral rods are provided that have predetermined curvatures to support various sections of the spine. The vertebral rods include a first end that is attached to a lower region of the spine, and a second end that is attached to an upper region of the spine. The construction of the rod results in a change in stiffness along the length between the first and second ends. The rods may also include predetermined curvatures to support various sections of the spine. The rods may include two of more longitudinal sections with different radii of curvatures to accommodate attachment to the various regions of the spine. The rods may also include one or more substantially straight sections. The rods may include a length to extend along a limited portion of the spine, or may extend along a majority of the spine. Further, the rods include a varying stiffness along the length. The stiffness generally decreases from the first end that is attached to a lower region of the spine to the second end that is attached to a higher region of the spine.

### BACKGROUND

The present application is directed to vertebral rods and, more particularly, vertebral rods with a predetermined shape that includes at least two sections each with different radii to facilitate placement along the spine and with a stiffness that changes from the first section to the second section.

The spine is divided into a variety of regions including the cervical, thoracic, and lumbar regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebrae that form the sacrum and the coccyx. The vertebrae of the spine are aligned in a curved configuration that includes a cervical curve, thoracic curve, and lumbosacral curve.

Various conditions may lead to damage of the intervertebral discs and the vertebral members. The damage may result from a variety of causes including a specific event such as trauma, a degenerative condition, a tumor, or infection. Damage to the intervertebral discs and vertebral members can lead to pain, neurological deficit, and/or loss of motion.

One or more vertebral rods are often used in a variety of different surgical treatments to treat these conditions. In some cases, vertebral rods are attached along the spine to facilitate spinal fusion to inhibit relative motion between vertebral members. In other cases, vertebral rods may be implanted with dynamic implants to preserve motion between vertebral members. Other treatments include implanting vertebral rods without the use of spinal fusion or dynamic implants.

Vertebral rods may provide a stable, rigid column that encourages bones to fuse after spinal-fusion surgery. Further, the rods may redirect stresses over a wider area away from a damaged or defective region. Also, the vertebral rods may restore the spine to its proper alignment. In the various surgical procedures, the vertebral rods may be attached to the exterior of two or more vertebral members, whether it is at a posterior, anterior, or lateral side of the spine.

### SUMMARY

The present application is directed to vertebral rods that are implanted along the spine of a patient. The rods may include first and second ends and an elongated shape configured to extend from the S1 vertebral member to at least the L2 vertebral member. The rods may include a longitudinal first section with a predetermined first radius configured to extend along the S1 and L5 vertebral members and may include a first stiffness. The rods may include a longitudinal second section positioned on an opposing side of the first section from the first end. The second section may have a different predetermined second radius that is larger than the first radius. The second section may have a greater length than the first section. The second section may have a smaller second stiffness. Each of the first and second radii may be positioned in a first direction. The rod may have a construction that prevents changing the first and second radii during the surgical procedure.

Another embodiment may include the rods having an elongated shape with a first end and an opposing second end. The rods may include a longitudinal first section with a predetermined first radius in a range of between about 20-60 mm. The rods may include a longitudinal second section positioned on an opposing side of the first section from the first end. The second section may have a predetermined second radius of between about 60-170 mm. Each of the first and second radii may be curved in a same direction. The rod may have a construction that prevents bending during the surgical procedure. The rod may have a stiffness that decreases from the first section to the second section.

Methods of implanting a vertebral rod within a patient are also included in the application. The methods may include implanting a polymeric rod along a spine of a patient. The rod may have a predetermined shape prior to the surgical procedure that includes a first radius and a different second radius. The rod may also include a first stiffness at the first radius and a smaller second stiffness at the second radius. The method may include positioning a first end of the rod at the sacrum. The method may include positioning a first section of the rod that includes the first radius along a lower lumbar section of the spine. The first radius may be in the range of between about 20-60 mm. The method may include positioning a second section of the rod that includes the second radius along a middle lumbar section of the spine. The second radius may be in the range of between about 60-170 mm. The method may also include securing the rod to the spine.

The various aspects of the various embodiments may be used alone or in any combination, as is desired.

### BRIEF DESCRIPTION OF THE DRAWINGS

FIG. 1 is a perspective view of a vertebral rod positioned along the vertebral members of the spine according to one embodiment.

FIG. 2 is a side view of a vertebral rod according to one embodiment.

FIG. 3 is a side view of a vertebral rod with first and second sections of different radii according to one embodiment.

FIG. 4 is a top view of a vertebral rod according to one embodiment.

FIG. 5 is a side view of a vertebral rod with first and second sections of different radii according to one embodiment.

FIG. 6 is a side view of a vertebral rod according to one embodiment.

FIG. 7 is a perspective view of a vertebral rod positioned along the vertebral members of the spine according to one embodiment.

FIG. 8 is a side view of a vertebral rod according to one embodiment.

FIG. 9 is a side view of a vertebral rod according to one embodiment.

### DETAILED DESCRIPTION

The present application is directed to vertebral rods sized to extend along the spine. The vertebral rods include a first end that is attached to a lower region of the spine, and a second end that is attached to an upper region of the spine. The construction of the rod results in a change in stiffness along the length between the first and second ends. The rods may also include predetermined curvatures to support various sections of the spine. The rods may include two of more longitudinal sections with different radii of curvatures to accommodate attachment to the various regions of the spine. The rods may also include one or more substantially straight sections. The rods may include a length to extend along a limited portion of the spine, or may extend along a majority of the spine. Further, the rods include a varying stiffness along the length. The stiffness generally decreases from the first end that is attached to a lower region of the spine to the second end that is attached to a higher region of the spine.

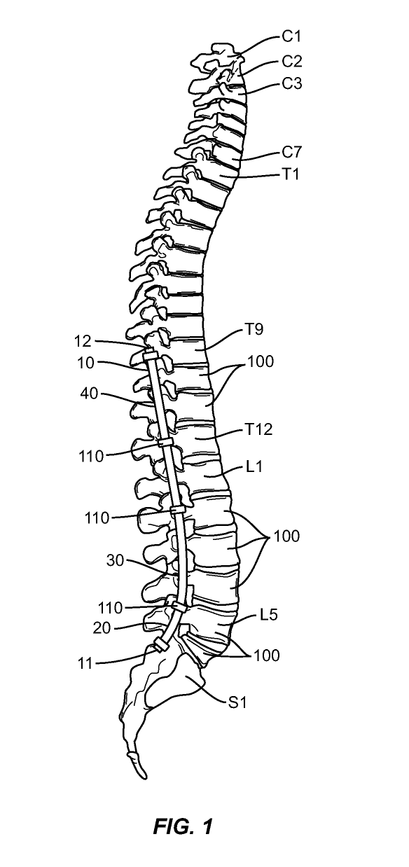

FIG. 1 schematically illustrates a vertebral rod 10 that spans from the S1 vertebral member 100 upwards along the spine. The rod 10 includes an elongated shape with opposing ends 11, 12. The rod 10 has a first longitudinal section 20 that extends along a first section of the spine and includes a first radius of curvature. In one embodiment, the first section 20 is positioned at the lower lumbar section of the spine and extends along the S1 and L5 vertebral members 100. The rod 10 also includes a second longitudinal section 30 that extends along a second section of the spine and includes a second radius of curvature. In one embodiment, the second section 30 is positioned at the middle lumbar section of the spine and extends along the L5-L2 vertebral members 100.

The rod 10 is attached to one or more vertebral members 100 by fasteners 110. Examples of fasteners 110 include bone screws, pedicle screws, and multi-axial screws. The fasteners 110 are well-known in the art and include a head with a channel sized to receive the rod 10 and a shaft section sized to extend into the vertebral members 100. The head may be fixedly positioned relative to the shaft, or may be movable relative to the shaft to accommodate the rod at various angular positions. The fasteners 110 may be coated with an osteoconductive material such as hydroxyapatite and/or osteoinductive agent such as a bone morphogenic protein for enhanced bony fixation.

The rod 10 includes a length to extend along the spine with the first end 11 positioned at the S1 vertebral member 110 and the second end 12 at a vertebral member 100 at a higher level of the spine. The rod 10 illustrated in FIG. 1 specifically includes a length for the second end 12 to be positioned at the T9 vertebral member 100. Another embodiment positions the second end 12 at the L2 vertebral member 100.

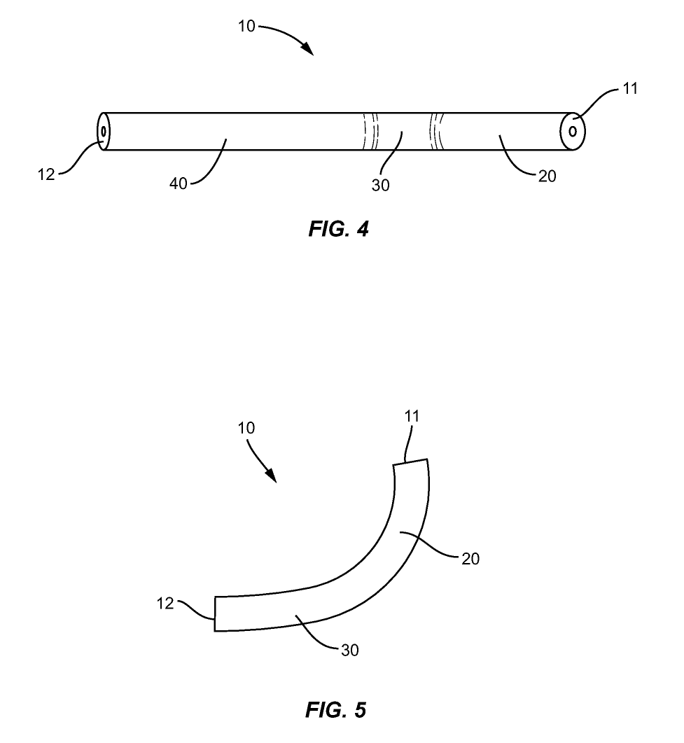

In one embodiment, the rod 10 includes an arcuate shape within a plane to accommodate the curvature of the spine. In one embodiment when the rod 10 is implanted in a patient, the curvature is mainly within the sagittal plane. The rod 10 is substantially straight when viewed from the top as illustrated in FIG. 4 with the sections 20, 30, 40 being laterally aligned.

The arcuate shape of the rod 10 is illustrated in the side view of FIG. 3. The rod 10 includes first, second, and third sections 20, 30, 40 positioned along the length between the ends 11, 12. The first section 20 is positioned at the first end 11 and extends inward along the rod 10 to a transition 15 with the second section 30. In one embodiment as illustrated in FIG. 3, the length X of the first section 20 measured from the centerline C of the at the first end 11 to the centerline C at the abutment face 15 is about 36 mm.

The first section 20 may extend from the first end 11 inward along the length of the rod 10. The first section 20 may also begin at a point inward from the first end 11. In these embodiments, a section of the rod 10 is positioned between the first section 20 and first end 11.

The second section 30 is positioned between the first and second ends 11, 12. The second section 30 extends between the transition 15 with the first section 20 to a transition 16 with the third section 40. In one embodiment, a length Y of the second section 30 measured from the centerline C between the transitions 15, 16. In one embodiment, the second section 30 is longer than the first section 20.

The third section 40 extends between the transition 16 and the second end 12. In one embodiment, the length Z of the third section is measured from the centerline C between the transition 16 and the second end 12.

The first section 20 includes a first radius R1 that accommodates the hyper lordotic curvature of the lower lumbar section of the spine along the S1 and L5 vertebral members 100. The first radius R1 may be in the range of between about 20-60 mm. In one specific embodiment, the first radius R1 is about 30 mm.

The second section 30 includes a different second radius R2 that accommodates the curvature of the spine through the middle lumbar region along the L5-L2 vertebral members 100. In some embodiments, the second radius R2 is larger than the first radius R1. The second radius R2 may be in the range of between about 60-170 mm. In one specific embodiment the second radius R2 is about 127 mm.

The third section 40 is substantially straight to accommodate at least the upper lumbar region along the L2-L1 vertebral members 100. The third section 40 may also include a length to extend along the lower thoracic spinal region including the T9-T12 vertebral members 100.

In one embodiment, the rod 10 does not include the third section 40. FIG. 5 includes an embodiment with the rod 10 having a first section 20 at the first end 11, and a second section 30 at the second end 12. This shorter version of the rod 10 is configured to span along a smaller section of the spine between the S1-L2 vertebral members 100. The length of the second section 30 may vary to extend farther along the spine to additional vertebral members 100 (e.g., the second end 12 terminates at the L1 vertebral member 100 or the T12 vertebral member 100). Alternatively, the length of the second section 30 may be shorter such that the rod 10 extends along fewer vertebral members 100 (e.g., the second end 12 terminates at the L3 vertebral member 100 or L4 vertebral member 100).

The embodiments described above include the first section 20 starting at the first end 11. Other embodiments may include the first section 20 spaced inward from the first end 11. A separate section of the rod 10 may be positioned between the first end 11 and the first section 20. This separate section may include various shapes including an arcuate shape with a different radius than the first radius R1 and a substantially straight shape. The length of the separate section may vary.

Likewise, in embodiments with third sections 40, the third sections 40 may be spaced away from the second end 12. A separate section of the rod 10 may extend beyond the third section 40 to be positioned along upper regions of the spine. The separate section may include various shapes and lengths.

The embodiments described above also include the first and second sections 20, 30 being abutted together the transition 15. Other embodiments may include the first and second sections 20, 30 being separated by an intermediate section. The intermediate section spaces apart the first and second sections 20, 30. The intermediate section may include various shapes and lengths. Likewise with embodiments having a third section 40, an intermediate section may be spaced between the third section 40 and the second section 30. The intermediate section may include various shapes and lengths. In some embodiments, the intermediate sections are substantially straight. In embodiments with intermediate sections between both the first and second sections 20, 30, and the second and third sections 30, 40, the intermediate sections may be the same or may be different.

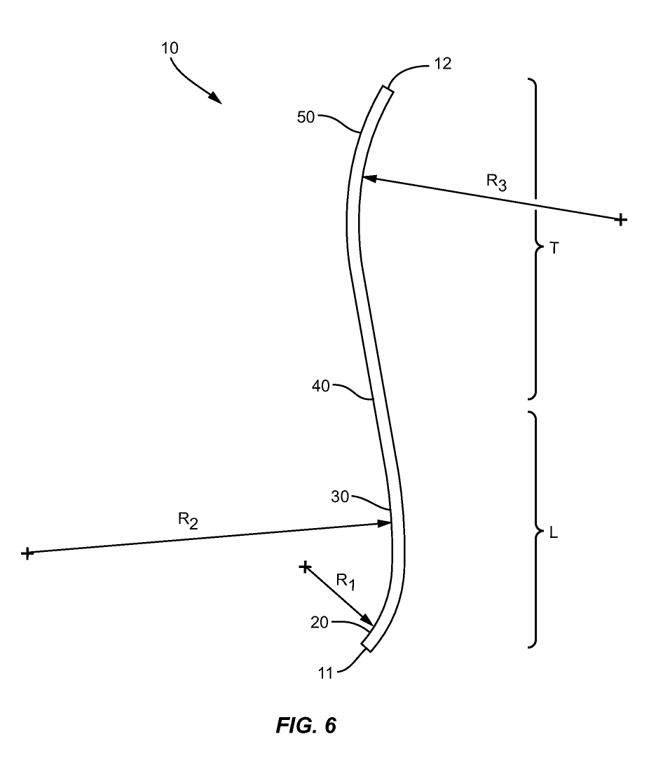

FIG. 6 includes a rod 10 with an upper section configured to support a thoracic section T of the spine. The rod 10 includes a length to extend along the spine with the first end 11 positioned at the S1 vertebral member 100 and the second end 12 positioned within the cervical section T. Embodiments include the second end 12 positioned at the T1 and T2 vertebral members 100. The rod 10 includes a variety of arcuate sections to accommodate the spine. The first section 20 includes a first radius R1 and the second section 30 includes a second radius R2 as described above. Section 50 includes a radius of curvature R3. Radius R3 is configured to accommodate the thoracic section T of the spine. The radius R3 is in an opposite direction from the first and second radii R1, R2 for section 50 to accommodate the kyphotic curvature along the thoracic section T with the sections 20, 30 curved to accommodate the lordotic curvature along the lumbar section L. In some embodiments, the radius R3 may be in the range of between about 100-200 mm. In one specific embodiment the second radius R3 is about 135 mm. The third section 40 is positioned between the sections 30, 40.

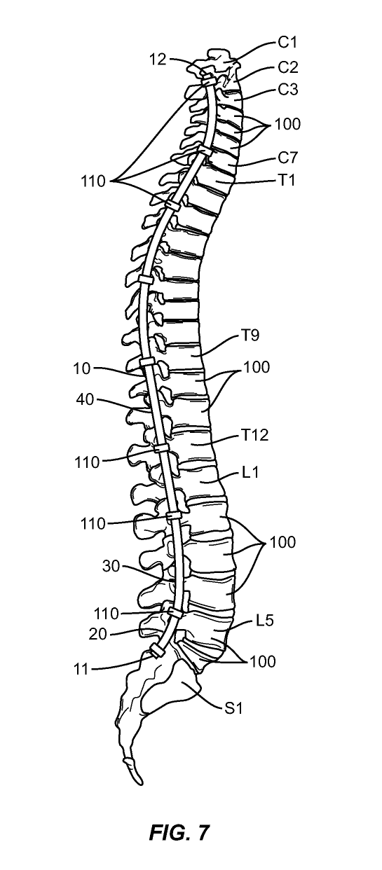

The rod 10 may also include a length to extend along a majority or entirety of the spine. FIG. 7 includes a rod 10 with a length to extend along the spine with the first end 11 positioned at the S1 vertebral member 100 and the second end 12 positioned at the C1 or C2 vertebral members 100. Fasteners 110 are positioned along the length to attach the rod 10 to the various vertebral members 100.

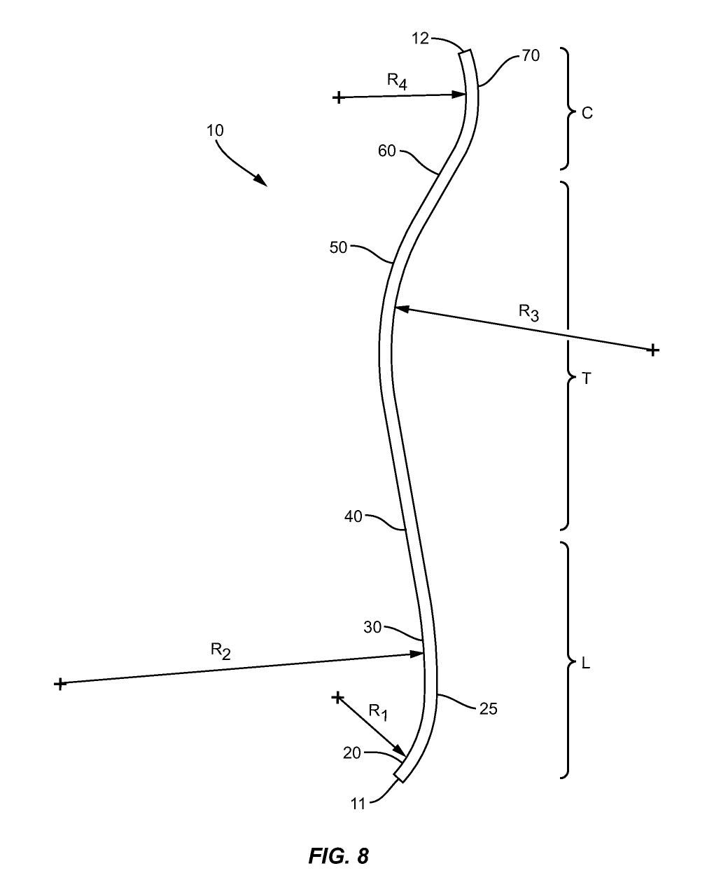

As illustrated in FIGS. 7 and 8, this rod 10 includes a variety of arcuate sections to accommodate the spine. The first section 20 includes a first radius R1 and the second section 30 includes a second radius R2. In this embodiment, an intermediate section 25 is positioned between the first and second sections 20, 30. This section of the rod 10 is configured to support the lumbar region L of the spine.

Section 50 is located upward along the spine and includes a radius of curvature R3. Radius R3 is configured to accommodate the thoracic region T of the spine. In some embodiments, the radius R3 may be in the range of between about 100-200 mm. In one specific embodiment the second radius R3 is about 135 mm.

The third section 40 is positioned between the sections 30, 50. The third section may be shaped including one or more arcuate sections or straight sections, to support one or both of the lumbar or thoracic regions L, T of the spine.

Section 70 is located to support the cervical region C. The section 70 may extend to the end 12, or may be spaced inward from the end 12. The radius R4 is established to accommodate the lordotic curvature of the cervical region C. The radius R4 is in the same direction as the first and second radii R1, R2 for section 50 to accommodate the lordotic curvature along the cervical region C. In some embodiments, the radius R4 may be in the range of between about 100-200 mm. In one specific embodiment the second radius R4 is about 135 mm.

Intermediate section 60 is positioned between and transitions the rod 10 from the curvature of section 50 to the curvature of section 70. Section 60 may include one or more curvatures and straight sections, to support one or both of the thoracic and cervical regions T, C.

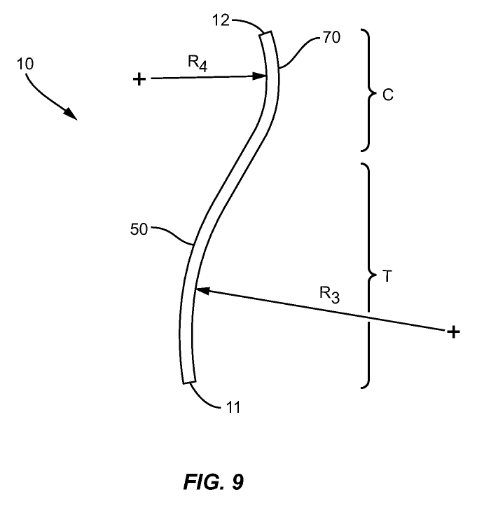

FIG. 9 includes an embodiment configured to support the thoracic and cervical regions T, C of the spine. Section 50 includes a radius of curvature R3 to accommodate the thoracic region T and section 60 includes a radius of curvature R4 to accommodate the cervical region C.

In the various embodiments, the rod 10 may have a length to extend across and support an entirety or just a limited portion of the spinal regions L, T, C.

The rod 10 is constructed such that the curvatures along the length cannot be changed during the surgical procedure. The rod 10 includes a predetermined shape that includes the various radii. The vertebral rod 10 can be fabricated from materials such as thermoplastics such as polyaryletherketone (PAEK) including PEEK and PEK, carbon-PEEK composites, biocompatible materials such as polymers including plastics, metals such as titanium, ceramics and composites thereof, rigid polymers including polyphenylene, polyamide, and polyimide. In one embodiment, the rod 10 is manufactured by injection molding. In a specific embodiment, the rod 10 is manufactured by injection molding using PEEK. The rod 10 may be constructed from the same material throughout the length, or may be constructed from two or more different materials along the length.

The rod 10 is constructed for the stiffness to vary along the length. The stiffness is greater towards the first end 11 and lesser towards the second end 12. Using the embodiment of FIGS. 7 and 8 as an example, the lumbar region L would have a high stiffness, the thoracic region T an intermediate stiffness, and the cervical region C a low stiffness.

In some embodiments, the stiffness may gradually decrease along the length of the rod 10. Other embodiments may be constructed for the change in stiffness to occur at discrete intervals along the length of the rod 10. One embodiment includes each section having the same construction and thus the same stiffness. In one embodiment, the transition in stiffness along the length of the rod 10 occurs within the intermediate sections and transitions 15, 16 between the sections.

The variable stiffness may be caused by the rod 10 having different constructions along the length of the rod 10. The different constructions may include different materials. In some embodiments, a lower section of the rod 10 is constructed from a first material, and an upper section 30 is constructed from a different second material. The materials may allow for the sections 20, 30 to be directly abutted together and fixed, or may require that the transition 15 provide for accommodating the different materials to connect the sections 20, 30 together.

The different constructions may also include different cross-sectional shapes and areas along the length. Examples of different cross-sectional shapes include but are not limited to circular, oval, and polygonal. The rod 10 may be solid, may include a hollow interior, or may include channel that is exposed to the exterior.

The rod 10 may also include combinations of different constructions to vary the stiffness along the length. By way of example, the first end 11 may be constructed from a first material and have a first cross-sectional shape, and the second end 12 may be constructed from a different material and have a different cross-sectional shape.

The rod 10 may also include radio-markers 60 along the length to determine the position of the rod 10 within the patient. Radio-markers 60 are included for identification under x-ray, fluoroscopy, CT or other imaging techniques. Metallic or ceramic radio-markers 60, such as tantalum beads, tantalum pins, titanium pins, titanium end caps and platinum wires can be positioned along the length of the rod 10.

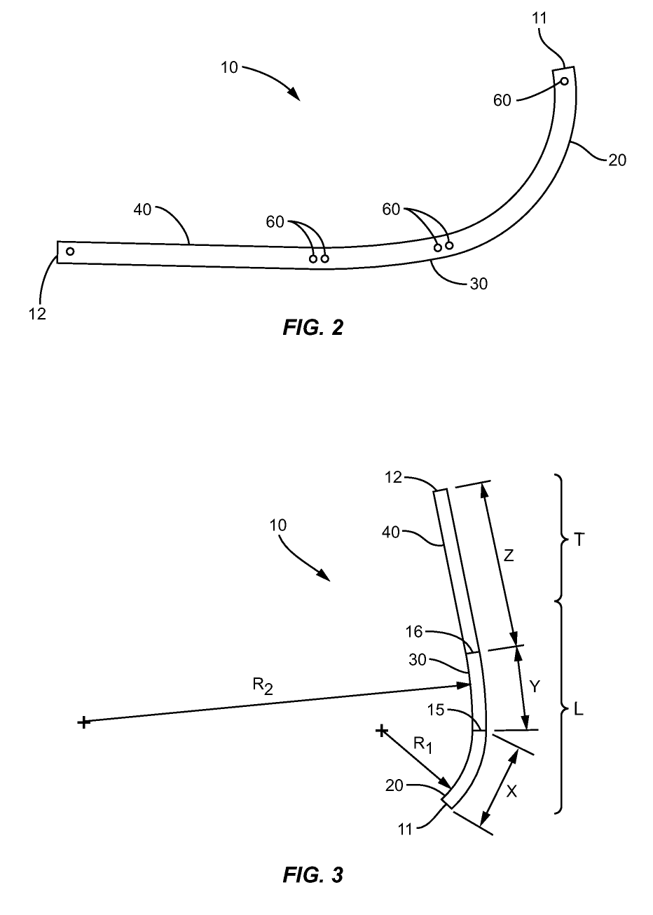

The radio-markers 60 may be positioned at the ends of one or more of the sections. FIG. 2 includes each sections 20, 30, 40 having a pair of radio-markers 60. A first marker 60 is positioned at a first end of the section and a second marker 60 is positioned at a second end of the section.

In use, the rod 10 includes a predetermined shape with the arcuate sections. The rod 10 includes a construction that prevents bending during the surgical procedure. The medical practitioner obtains access to the spine in any appropriate manner, including open surgery, mini-open surgery, minimally invasive surgery and percutaneous surgical implantation, whereby the vertebral members 100 are accessed through a micro-incision, or sleeve that provides a protected passageway to the area.

The rod 10 is implanted along the spine with at least the first and second sections being aligned with the appropriate sections of the spine. The first end 11 of the rod 10 is positioned at a lower region of the spine, such as at the S1 vertebral member 100. The various sections of the rod 10 are aligned with the spinal regions. In one embodiment, the first section 20 is aligned along the lower lumbar region of the spine, the second section 30 is aligned along a middle lumbar region of the spine, and the third section 40 is aligned along the upper lumbar region and may also aligned along the lower thoracic region depending upon the length of the section 40.

Once the rod 10 is properly positioned along the spine, the rod 10 is secured with one or more fasteners 110 to one or more of the vertebral members 100.

In some procedures, a single rod 10 is implanted along the spine. Other embodiments may include a pair of rods 10 implanted along the spine with a first rod 10 on a first lateral side of the vertebral members 100, and a second rod 10 on a second lateral side.

The rod 10 may be used within a living patient for the treatment of various spinal disorders. The rod 10 may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A vertebral rod for use in a surgical procedure, the rod comprising:
an elongated shape having a first end and an opposing second end and a length measured between the first and second ends configured to extend from the S1 vertebral member to at least the L2 vertebral member;
a longitudinal first section having a predetermined first radius configured to extend along the S1 and L5 vertebral members, the first section having a first stiffness;
a longitudinal second section positioned on an opposing side of the first section from the first end, the second section having a different predetermined second radius that is larger than the first radius, the second section having a greater length than the first section, the second section having a smaller second stiffness;
each of the first and second radii being positioned in a first direction;
the rod having a construction that prevents changing the first and second radii during the surgical procedure.

2. The vertebral rod of claim 1, wherein the first radius is in a range of between about 20-60 mm.

3. The vertebral rod of claim 1, wherein the second radius is in a range of between about 60-170 mm.

4. The vertebral rod of claim 1, wherein the first section extends from the first end inward towards the second end.

5. The vertebral rod of claim 1, further comprising a longitudinal third section positioned on an opposing side of the second section from the first end, the third section being substantially straight and having a lower stiffness than the first and second sections.

6. The vertebral rod of claim 1, wherein further comprising a thoracic section positioned on an opposing side of the second section from the first section, the thoracic section having a third radius directed in an opposite direction from the first and second radii, the third radius being different than the first and second radii, the thoracic section having a different construction and a lower stiffness than the first and second sections.

7. The vertebral rod of claim 6, further comprising a cervical section positioned on an opposing side of the thoracic section from the first section, the cervical section having a fourth radius directed in the same direction as the first and second radii, the fourth radius being different than the first, second, and third radii, the cervical section having a different construction and a lower stiffness than the first, second, and third sections.

8. The vertebral rod of claim 1, wherein a stiffness of the rod decreases along the length from the first end to the second end.

9. A vertebral rod for use in a surgical procedure, the rod comprising:
an elongated shape having a first end and an opposing second end;
a longitudinal first section having a predetermined first radius in a first range of between about 20-60 mm;
a longitudinal second section positioned on an opposing side of the first section from the first end, the second section having a predetermined second radius in a second range of between about 60-170 mm;
each of the first and second radii being curved in a same direction;
the rod having a construction that prevents bending during the surgical procedure;
the rod further including a stiffness that decreases from the first section to the second section.

10. The vertebral rod of claim 9, wherein each of the first section includes a first construction with a first stiffness and the second section includes a different second construction with a smaller second stiffness.

11. The vertebral rod of claim 9, wherein the second section has a greater length than the first section.

12. The vertebral rod of claim 9, wherein the first section starts at the first end.

13. The vertebral rod of claim 9, further comprising a longitudinal third section positioned on an opposing side of the second section from the first end, the third section having a different construction and a smaller stiffness than the first and second sections.

14. The vertebral rod of claim 9, further comprising a cervical section at the second end, the cervical section having a lower stiffness and a different construction than the first, second, and third sections.

15. The vertebral rod of claim 9, further comprising a longitudinal intermediate section positioned between the first and second sections, the intermediate section including a different shape than the first and second sections.

16. The vertebral rod of claim 9, wherein the rod is constructed from PEEK.

17. A method of implanting a vertebral rod within a patient in a surgical procedure comprising:
implanting a polymeric rod along a spine of a patient, the rod having a predetermined shape prior to the surgical procedure that includes a first radius and a different second radius, the rod also including a first stiffness at the first radius and a smaller second stiffness at the second radius;
positioning a first end of the rod at the sacrum;
positioning a first section of the rod that includes the first radius along a lower lumbar section of the spine, the first radius being in the range of between about 20-60 mm;
positioning a second section of the rod that includes the second radius along a middle lumbar section of the spine, the second radius being in the range of between about 60-170 mm; and
securing the rod to the spine.

18. The method of claim 17, further comprising positioning a third section of the rod along an upper lumbar section of the spine, the third section being substantially straight.

19. The method of claim 17, wherein positioning the first section of the rod that includes the first radius along the lower lumbar section of the spine includes positioning the first section along the S1 vertebral member and the L5 vertebral member.

20. The method of claim 17, further comprising positioning an intermediate section of the rod between the first and second sections along the spine.
